(19) United States
(12) Patent Application Publication
(10) Pub. No.: US 2009/0182022 A1
(43) Pub. Date: Jul. 16, 2009

(54) Treatment of Fatty Liver

(75) Inventors: Roelof M. L. Rongen, Califon, NJ (US); Robert A. Shalwitz, Bexley, OH (US); Ihor Terleckyj, Bloomfield, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Reliant Pharmaceuticals

(21) Appl. No.: 12/087,411

(22) PCT Filed: Jan. 5, 2007
(86) PCT No.: PCT/US2007/000224

§ 371 Date: Dec. 5, 2008

Related U.S. Application Data
(60) Provisional application No. 60/756,226, filed on Jan. 5, 2006.
(60) Provisional application No. 60/836,976, filed on Aug. 11, 2006.

(51) Int. Cl. A61K 31/20; A61K 31/426; A61P 1/16

(52) U.S. Cl. 514/365; 514/560

(57) ABSTRACT

Methods and compositions comprising peroxisomal and/or mitochondrial beta oxidation stimulating agents to reverse or resolve, slow the progression of, treat or prevent the development of fatty liver and conditions stemming from fatty liver, such as NASH, liver inflammation, cirrhosis and liver failure. An active agent that by itself is associated with an increased risk of fatty liver development and conditions stemming from fatty liver, such as NASH, liver inflammation, cirrhosis and liver failure, may be administered in combination with peroxisomal and/or mitochondrial beta oxidation stimulating agents. A combination regimen involving such agents, as simultaneous or concomitant therapy, or as a fixed dosage form, is also provided.

### RELATED APPLICATION DATA

This application claims priority from U.S. Provisional Application No. 60/756,226, which was filed on Jan. 5, 2006, and U.S. Provisional Application No. 60/836,976, which was filed on Aug. 11, 2006, the contents of which are incorporated herein by reference.

### BACKGROUND OF THE INVENTION

1. Field of the Invention

The present invention relates to methods and compositions comprising peroxisomal and/or mitochondrial beta oxidation stimulating agents for the treatment or prevention of fatty liver and conditions stemming from fatty liver, such as NASH, liver inflammation, cirrhosis and liver failure.

2. Description of the Related Art

Fatty liver, i.e., steatosis, is a disease in which excessive amounts of lipids accumulate in the liver. Fatty liver may develop due to medicine or alcohol use, viral (e.g., Hepatitis C) or bacterial infections or obesity. Steatohepatitis is inflammation of the liver related to fat accumulation. Heavy alcohol use can lead to fatty liver and inflammation and is usually referred to as alcoholic hepatitis. Steatohepatitis resembles alcoholic hepatitis, but can occur in people who seldom or never drink alcohol. In this instance, it is often called nonalcoholic steatohepatitis or NASH. Both alcoholic hepatitis and steatohepatitis can lead to scarring, e.g., cirrhosis, and hardening of the liver resulting in serious liver damage.

There are reported to be over 1,000 drugs and chemicals that are capable of causing injury to the liver. The term drug-induced liver disease is used to describe those instances in which an active agent has caused injury to the liver. Drug-induced liver injury may account for as many as 10 percent of hepatitis cases in adults overall, 40 percent of hepatitis cases in adults over fifty years old, and 25 percent of cases of fulminant liver failure. Certain active agents, such as glucocorticoids, synthetic estrogens, amiodarone, tamoxifen and valproic acid, for example, have been associated with fatty liver.

Thus, there is a need for methods and compositions for the treatment and prevention of the development of fatty liver and conditions stemming from fatty liver, such as NASH, liver inflammation, cirrhosis and liver failure.

Omega-3 fatty acids are known to reduce serum triglycerides by inhibiting DGAT and by stimulating peroxisomal and mitochondrial beta oxidation. Two omega-3 fatty acids, eicosapentaenoic acid (EPA) and docosahexaenoic acid (DHA), have been found to have high affinity for both PPAR-alpha and PPAR-gamma. Marine oils, e.g., fish oils, are a good source of EPA and DHA, which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One such form of omega-3 fatty acid is a concentrate of omega-3, long chain, polyunsaturated fatty acids from fish oil containing DHA and EPA and is sold under the trademark Omacor®. Such a form of omega-3 fatty acid is described, for example, in U.S. Pat. Nos. 5,502,077, 5,656,667 and 5,698,594, the disclosures of which are incorporated herein by reference.

Peroxisome proliferator-activated receptors (PPARs) are members of the nuclear hormone receptor superfamily of ligand-activated transcription factors that are related to retinoid, steroid and thyroid hormone receptors. There are three distinct PPAR subtypes that are the products of different genes and are commonly designated PPAR-alpha, PPAR-beta/delta (or merely, delta) and PPAR-gamma. General classes of pharmacological agents that stimulate peroxisomal activity are known as PPAR agonists, e.g., PPAR-alpha agonists, PPAR-gamma agonists and PPAR-delta agonists. Some pharmacological agents are combinations of PPAR agonists, such as alpha/gamma agonists, etc., and some other pharmacological agents have dual agonist/antagonist activity.

Fibrates such as fenofibrate, bezafibrate, clofibrate and gemfibrozil, are PPAR-alpha agonists and are used in patients to decrease lipoproteins rich in triglycerides, to increase HDL and to decrease atherogenic-dense LDL. Fibrates are typically orally administered to such patients.

Fenofibrate or 2-[4-(4-chlorobenzoyl)phenoxy]-2-methyl-propanoic acid, 1-methylethyl ester, has been known for many years as a medicinally active principle because of its efficacy in lowering blood triglyceride and cholesterol levels. Fenofibrate is very poorly soluble in water and the absorption of fenofibrate in the digestive tract is limited. Treatment with 40 to 300 mg of fenofibrate per day enables a 20 to 25% reduction of cholesterolemia and a 40 to 50% reduction of triglyceridemia to be obtained.

PPAR-gamma agonists, such as the thiazolidinediones (e.g., troglitazone, pioglitazone and rosiglitazone), and partial PPAR-gamma agonist/antagonists, such as metaglidasen, are used for the treatment of type II diabetes.

Capanni et al., Aliment Pharmacol Ther 3:1143-51 (2006), describes a human trial intended to evaluate the effects of administering a 1 g capsule containing n-3 PUFAs (eicosapentaenoic acid:docosahexaenoic acid=0.9:1.5) daily for twelve months to patients suffering from non-alcoholic fatty liver disease. The authors observed reductions in serum aspartate transaminase, alanine transaminase, y-glutamyl transpeptidase, triglycerides, and fasting glucose, as well as improved liver echotexture (assessed by ultrasonography) and improved liver blood flow (assessed by echo-Doppler). Although not conclusive, Capanni et al. indicated that these results warranted further evaluation via a large randomized, double-blind, placebo-controlled trial using liver histology as an endpoint.

Puder et al., U.S. Patent Publication No. 2006/0127491, is directed to a method of treating or preventing parenteral nutrition induced liver disease by administering to a patient an intravenous emulsion containing fish oil enriched in omega-3 fatty acid triglycerides, for a period of at least three weeks. According to Use described therein, the patient must not be administered phytosterols or plant-derived fatty acids. Puder et al. does not contemplate the oral use of peroxisomal and/or mitochondrial beta oxidation stimulating agents, such as omega-3 fatty acids, for the treatment and prevention of the development of fatty liver and conditions stemming from fatty liver, such as liver inflammation, cirrhosis and liver failure, as part of a chronic dosing regimen on an out-patient basis.

There is a need in the art for methods and compositions for the treatment and prevention of the development of fatty liver and conditions stemming from fatty liver, such as liver inflammation, cirrhosis and liver failure.

### SUMMARY OF THE INVENTION

The present invention relates to methods and compositions comprising peroxisomal and/or mitochondrial beta oxidation stimulating agents to reverse or resolve, slow the progression of, treat or prevent the development of fatty liver and conditions stemming from fatty liver, such as NASH, liver inflammation, cirrhosis and liver failure.

One embodiment of the present invention provides methods of utilizing a PPAR agonist or dual agonist/antagonist, a peroxisomal and/or mitochondrial beta oxidation stimulating agent, or combinations of any of these types of compounds, to reverse or resolve, slow the progression of, treat or prevent the development of fatty liver and conditions stemming from fatty liver, such as NASH, liver inflammation, cirrhosis and liver failure.

Another embodiment of the present invention provides pharmaceutical compositions comprising a PPAR agonist or dual agonist/antagonist, a peroxisomal and/or mitochondrial beta oxidation stimulating agent, or combinations thereof. In one aspect of the embodiment, the compositions of the present invention are used to reverse or resolve, slow the progression of, treat or prevent the development of fatty liver and conditions stemming from fatty liver, such as NASH, liver inflammation, cirrhosis and liver failure.

In other embodiments, the present invention provides administering an active agent that by itself is associated with an increased risk of fatty liver development and conditions stemming from fatty liver, such as NASH, liver inflammation, cirrhosis and liver failure, in combination with a PPAR agonist or dual agonist/antagonist, a peroxisomal and/or mitochondrial beta oxidation stimulating agent, or combinations thereof, to reverse or resolve, slow the progression of, treat or prevent the development of fatty liver and conditions stemming from fatty liver, such as NASH, liver inflammation, cirrhosis and liver failure. The invention also provides a combination regimen involving such agents, as simultaneous or concomitant therapy, or as a fixed dosage form.

Other novel features and advantages of the present invention will become apparent to those skilled in the art upon examination of the following or upon learning by practice of the invention.

### DESCRIPTION OF THE PREFERRED EMBODIMENTS

Uses and compositions of the present invention may be used to treat or prevent all types of fatty liver disease including, but not limited to, medication induced fatty liver (e.g., HIV patients on HAART therapy), alcohol induced fatty liver, viral and bacterial infection induced fatty liver (e.g., Hepatitis C) and obesity induced fatty liver, as well as conditions stemming from fatty liver, such as NASH, liver inflammation, cirrhosis and liver failure.

According to one embodiment of the present invention, Uses and compositions comprise a peroxisomal and/or mitochondrial beta oxidation stimulating agent to reverse or resolve, slow the progression of, treat or prevent the development of fatty liver and conditions stemming from fatty liver, such as NASH, liver inflammation, cirrhosis and liver failure. For example, such agents include, but are not limited to, omega-3 fatty acids, a PPAR agonist or dual agonist/antagonist, or combinations thereof. As used herein, the term “PPAR agonists or dual agonist/antagonist” includes, but is not limited to, PPAR-alpha, PPAR-gamma, PPAR-delta, PPAR-alpha/gamma, PPAR-gamma/delta, PPAR-alpha/delta, and PPAR-alpha/gamma/delta agonists and dual agonists/antagonists. The present invention may incorporate now known or future known PPAR agonists or dual agonists/antagonists in an amount generally recognized as safe. Specific PPAR agonists or dual agonists/antagonists include, but are not limited to, the fibrates, the thiazolidinediones, the non-thiazolidinediones and metaglidasen. Preferably, the PPAR agonist or dual agonist/antagonist is a fibrate, such as fenofibrate, bezafibrate, clofibrate and gemfibrozil, most preferably fenofibrate.

In some embodiments, the present invention provides the use of a PPAR agonist or dual agonist/antagonist to reverse or resolve, slow the progression of, treat or prevent the development of fatty liver and conditions stemming from fatty liver, such as NASH, liver inflammation, cirrhosis and liver failure. In other embodiments, the present invention provides the use of peroxisomal and/or mitochondrial beta oxidation stimulating agents, such as but not limited to omega-3 fatty acids, to reverse or resolve, slow the progression of, treat or prevent the development of fatty liver and conditions stemming from fatty liver, such as NASH, liver inflammation, cirrhosis and liver failure.

In still other embodiments, the present invention provides a pharmaceutical composition suitable to reverse or resolve, slow the progression of, treat or prevent the development of fatty liver and conditions stemming from fatty liver, such as NASH, liver inflammation, cirrhosis and liver failure, comprising a PPAR agonist or dual agonist/antagonist, a peroxisomal and/or mitochondrial beta oxidation stimulating agent, or combinations thereof. In a further embodiment, the pharmaceutical composition of the invention is administered simultaneous to administration of an active agent that by itself is associated with an increased risk of fatty liver development and conditions stemming from fatty liver, such as NASH, liver inflammation, cirrhosis and liver failure, e.g., as a single fixed dosage form or as separate pharmaceutical compositions administered at the same time. In other embodiments, the active agent that by itself is associated with an increased risk of fatty liver development is administered apart from the pharmaceutical composition of the invention, but the therapy is concomitant. For example, the active agent that by itself is associated with an increased risk of fatty liver development may be administered weekly with daily intake of the pharmaceutical composition of the invention, or the components can be administered at different times on the same day. One skilled in the art with the benefit of the present disclosure will understand that the precise dosage and schedule for the administration of the several active agents will vary depending on numerous factors, such as, for example, the route of administration and the seriousness of the condition. The most preferred route of administration for the compositions of the present invention is per os (oral), either in a solid or liquid form, or a combination thereof.

The peroxisomal and/or mitochondrial beta oxidation stimulating agent preferably comprises omega-3 fatty acids. As used herein, the term “omega-3 fatty acids” includes natural or synthetic omega-3 fatty acids, or pharmaceutically acceptable esters, derivatives, conjugates (See, e.g., Zaloga et al., U.S. Patent Application Publication No. 2004/0254357, and Horrobin et al., U.S. Pat. No. 6,245,811, the disclosures of which are hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified oil concentrates from marine animal origin, e.g. fish or krill. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if other forms are utilized in accordance with the present invention.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acids can be present in an amount from about 350 mg to about 10 grams, more preferably about 500 mg to about 6 grams, and most preferably from about 750 mg to about 4 grams. This amount may be in one or more dosage forms, preferably one dosage form. The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids are the Omacor® omega-3 fatty acids (K85EE, Pronova Biocare A.S., Lysaker, Norway), which preferably comprise the following characteristics (per dosage form):

| Test | Minimum Value | Maximum Value |
|---|---|---|
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n-3 fatty acids | 90% (w/w) | |

The single fixed dosage form comprising a PPAR agonist or dual agonist/antagonist, a peroxisomal and/or mitochondrial beta oxidation stimulating agent, or combinations thereof, and an active agent that by itself is associated with an increased risk of fatty liver development and conditions stemming from fatty liver, such as NASH, liver inflammation, cirrhosis and liver failure, may be administered in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin.

The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Excipients may include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The daily dosages of PPAR agonist or dual agonist/antagonist, peroxisomal and/or mitochondrial beta oxidation stimulating agent, or combinations thereof, may be determined by those of ordinary skill in the art depending on subject age, gender, seriousness of the condition, etc. The daily dosages may be administered in from 1 to 10 dosages, with the preferred number of dosages from 1 to 4 times a day to deliver a total dose for any single agent between 1 mg and 8000 mg per day. The administration is preferably oral administration.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure. All references cited herein are hereby incorporated by reference in their entirety.

While the present invention has been described for what are presently considered the preferred embodiments, the invention is not so limited. To the contrary, the invention is intended to cover various modifications and equivalent arrangements included within the spirit and scope of the detailed description provided above.

1-44. (canceled)

45. A method of administering to a patient an active agent associated with an increased risk of fatty liver disease, NASH, liver inflammation, cirrhosis, or liver failure, comprising administering the active agent in combination with at least one peroxisomal and/or mitochondrial beta oxidation stimulating agent which is provided in an amount effective for treating and/or preventing the development of fatty liver disease, NASH, liver inflammation, cirrhosis, or liver failure.

46. The method of claim 45, wherein the at least one peroxisomal and/or mitochondrial beta oxidation stimulating agent is administered apart from administration of the active agent associated with an increased risk of fatty liver disease, NASH, liver inflammation, cirrhosis, or liver failure.

47. The method of claim 45, wherein the at least one peroxisomal and/or mitochondrial beta oxidation stimulating agent is administered simultaneous to administration of the active agent associated with an increased risk of fatty liver disease, NASH, liver inflammation, cirrhosis, or liver failure.

48. The method of claim 45, wherein the at least one peroxisomal and/or mitochondrial beta oxidation stimulating agent is administered in unit dosage form with the active agent associated with an increased risk of fatty liver disease, NASH, liver inflammation, cirrhosis, or liver failure.

49. The method of claim 45, wherein the at least one peroxisomal and/or mitochondrial beta oxidation stimulating agent comprises omega-3 fatty acids.

50. The method of claim 45, wherein the omega-3 fatty acids are present in a concentration of at least 40% by weight as compared to the total fatty acid content of the composition.

51. The method of claim 45, wherein the omega-3 fatty acids are present in a concentration of at least 80% by weight as compared to the total fatty acid content of the composition.

52. The method of claim 45, wherein the omega-3 fatty acids comprise at least 50% by weight of EPA and DHA as compared to the total fatty acid content of the composition.

53. The method of claim 45, wherein the omega-3 fatty acids comprise at least 80% by weight of EPA and DHA as compared to the total fatty acid content of the composition.

54. The method of claim 45, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

55. A method of treating and/or preventing the development of fatty liver disease and/or conditions related thereto, comprising the step of administering to a subject a therapeutically effective amount of a composition comprising at least one peroxisomal and/or mitochondrial beta oxidation stimulating agent and optionally further comprising at least one PPAR agonist or dual agonist/antagonist.

56. The method of claim 55, wherein the conditions related to fatty liver disease are selected from the group consisting of NASH, liver inflammation, cirrhosis, and liver failure.

57. The method of claim 55, wherein the at least one peroxisomal and/or mitochondrial beta oxidation stimulating agent comprises omega-3 fatty acids.

58. The method of claim 57, wherein the omega-3 fatty acids are present in a concentration of at least 40% by weight as compared to the total fatty acid content of the composition.

59. The method of claim 57, wherein the omega-3 fatty acids are present in a concentration of at least 80% by weight as compared to the total fatty acid content of the composition.

60. The method of claim 57, wherein the omega-3 fatty acids comprise at least 50% by weight of EPA and DHA as compared to the total fatty acid content of the composition.

61. The method of claim 57, wherein the omega-3 fatty acids comprise at least 80% by weight of EPA and DHA as compared to the total fatty acid content of the composition.

62. The method of claim 57, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

63. The method of claim 55, wherein the at least one PPAR agonist or dual agonist/antagonist is selected from the group consisting of a fibrate, a thiazolidinedione, a non-thiazolidinedione and metaglidasen.

64. The method of claim 55, wherein the PPAR agonist or dual agonist/antagonist comprises a fibrate.
